(19) United States
(12) Patent Application Publication
(10) Pub. No.: US 2026/0132381 A1
(43) Pub. Date: May 14, 2026

(54) METHOD AND SYSTEM FOR ORGAN-ON-A-CHIP DESIGN BASED ON SIMILARITY PRINCIPLE, AND ORGAN-ON-CHIPS

(71) Applicant: QILU UNIVERSITY OF TECHNOLOGY (SHANDONG ACADEMY OF SCIENCES), Jinan (CN)

(72) Inventors: Weiguang SU, Jinan (CN); Yang ZHAO, Jinan (CN); Chenxu BI, Jinan (CN); Li WANG, Jinan (CN); Jun CHEN, Jinan (CN)

(21) Appl. No.: 19/223,165

(22) Filed: May 30, 2025

(30) Foreign Application Priority Data
Nov. 12, 2024 (CN) 2024116037372

(51) Int. Cl. C12N 5/071; G16B 5/00

(52) U.S. Cl. CPC C12N 5/0671; C12N 5/0679; G16B 5/00; C12N 2513/00

(57) ABSTRACT

Provided is a method for designing organ-on-chips (OoCs) based on similarity principle, including determining a scaling ratio to obtain geometric dimensions of cell culture region of OoC; selecting dimensionless number as parameter calculation basis of the OoC, and calculating a range of corresponding in-vivo dimensionless number; acquiring flow rate range of fluid and size range of channels in OoC based on shear stress on cells in organ and calculated range of the dimensionless number; calculating characteristic lengths of channels according to the scaling ratio and determining corresponding flow rate; determining outer wall thickness of OoC based on oxygen permeability of material used for the OoC; performing simulation to verify whether all parameter indicators of OoC meet design requirements, and making mold sets according to outputted size design parameters for preparing the OoC; and, performing PDMS casting, curing, demolding, and bonding by using the mold sets to obtain the finished OoC.

### CROSS-REFERENCE TO RELATED APPLICATIONS

The present invention claims priority benefits to Chinese Patent Application No. 202411603737.2, filed with the China National Intellectual Property Administration on Nov. 12, 2024 and entitled “A SIMILARITY PRINCIPLE-BASED DESIGN METHOD OF ORGAN-ON-A-CHIP AND SYSTEM THEREOF, AND ORGAN-ON-CHIPS”, which is incorporated herein by reference in its entirety and constitutes a part of the present invention for all purposes.

### TECHNICAL FIELD

The present invention relates to the field of organ-on-chips (OoCs) technologies, and specifically, to a method for designing organ-on-a-chip (OoC) based on the similarity principle, a system therefor, and organ-on-chips.

### BACKGROUND

OoCs provide a powerful in-vitro platform for studying the physiology, pathology, and pharmacology of organs. For example, absorption of drugs can be studied by using gut-on-a-chip (GoC), mechanisms of alcoholic liver injury can be studied by using liver-on-a-chip (LoC), and first-pass metabolism of drugs can be studied by using gut-liver system on chips.

However, it is difficult for existing OoCs to simultaneously reproduce multiple physiological characteristics of a human organ, such as a geometric shape, a fluid dynamic environment, and an oxygen gradient, consequently affecting the reliability of experimental results. Sizes and parameters of designed OoCs vary due to different focuses. A GoC design is used as an example. Viscosity of chyme in the intestine is a complex parameter that varies according to various factors, such as food composition, digestion stages, and individual differences. Based on existing studies on the viscosity of chyme under different conditions, the Reynolds number of the human intestine, through calculation, ranges from 0.125 to 70. However, in some existing studies on GoC, the height of an upper channel of a GoC is set to 500 μm to establish an oxygen gradient in the GoC similar to that in vivo, but in this case, the Reynolds number in the upper channel is only 0.0185. In some other studies, a GoC is designed to reproduce a shear stress of 0.02 dyne/cm² on intestinal epithelial cells, but in this case, the Reynolds number in the channel is 0.0395. The Reynolds numbers of the GoC are far different from those of the human intestine in actual situations.

It can be learned that, in the related art, different research parameters are designed for different purposes, but result in Reynolds numbers far different from the actual intestinal Reynolds number range, making it difficult to realistically reproduce a fluid dynamic environment in the intestine. Similar problems exist in other OoCs, making it difficult to simultaneously reproduce multiple physiological characteristics of an organ in experiments.

### SUMMARY

To resolve the deficiencies in the related art, an objective of the present invention is to provide a method for designing OoC based on the similarity principle, a system therefor, and OoCs, to make the finished OoC closer to a real state in a human body.

To achieve the foregoing objective, a first aspect of the present invention provides a method for designing OoC based on the similarity principle, including:
determining a scaling ratio of an OoC to a human organ, and obtaining a geometric dimension of a cell culture region of the OoC;
selecting a dimensionless number as a parameter calculation basis of the OoC, and calculating a range of a corresponding dimensionless number in an in-vivo organ;
acquiring a flow rate range of a fluid and a size range of a channel in the OoC based on a shear stress on cells in the human organ and the calculated range of the dimensionless number;
calculating a characteristic length of the channel and determining a corresponding flow rate according to the scaling ratio;
determining an outer wall thickness of the OoC based on the oxygen permeability of a material used for the OoC;
performing simulation by using a computational fluid dynamics tool to verify whether all parameter indicators of the OoC meet design requirements, and outputting a channel characteristic length design parameter and an outer wall thickness design parameter that meet the design requirements;
making a mold set according to the channel characteristic length design parameter and the outer wall thickness design parameter that are outputted through calculation, for preparing the OoC; mixing polydimethylsiloxane (PDMS) and a curing agent at a specific ratio, degassing the mixture under vacuum, injecting the mixture into the mold set for preparing the OoC, heating the mold set for curing, and then performing demolding to obtain multiple layers of PDMS substrates including a channel structure, and making connection holes; and performing plasma treatment on an outer wall layer of the multiple layers of the PDMS substrates and on a porous membrane, applying a PDMS adhesive around a channel, and aligning, bonding, and curing the multiple layers of the PDMS substrates following a OoC structure to finally complete an assembly of the OoC that meets the design requirements.

A second aspect of the present invention provides a system for designing OoC based on the similarity principle, including:
a geometric similarity module, configured to determine a scaling ratio of an OoC to a human organ, and obtain a geometric dimension of a cell culture region of the OoC;
a dimensionless number selection module, configured to select a dimensionless number as a parameter calculation basis of the OoC, and calculate a range of a corresponding dimensionless number in an in-vivo organ;
a flow rate and channel size calculation module, configured to acquire a flow rate range of a fluid and a size range of a channel in the OoC based on a shear stress on cells in the human organ and the calculated range of the dimensionless number;
a characteristic length calculation module, configured to calculate a characteristic length of the channel and determine a corresponding flow rate according to the scaling ratio;
an outer wall thickness calculation module, configured to determine an outer wall thickness of the OoC based on oxygen permeability of a material used for the OoC; and
a verification module, configured to perform simulation to verify whether all parameter indicators of the OoC meet design requirements, and output a channel characteristic length design parameter and an outer wall thickness design parameter that meet the design requirements;
wherein, making a mold set for preparing the OC according to the channel characteristic length design parameter and the outer wall thickness design parameter that are outputted through calculation, comprising: mixing PDMS and a curing agent at a specific ratio, degassing under vacuum and injecting the mixture into the mold set for preparing the OoC, heating the mold set for curing, then obtaining multiple layers of PDMS substrates including a multi-channel structure after demolding, and making connection holes thereon; and performing plasma treatment on an outer wall layer of the multiple layers of the PDMS substrates and a porous membrane, applying a PDMS adhesive around a channel, and aligning, bonding and curing the multiple layers of the PDMS substrates following a OoC structure to finally complete an assembly of the OC that meets the design requirements.

A third aspect of the present invention provides an OoC being obtained based on the method for designing OoC based on the similarity principle provided according to the first aspect. A plurality (a set of) of molds for preparing the OoC are made according to the channel characteristic length design parameter and the outer wall thickness design parameter that are outputted through calculation;
mixing PDMS and a curing agent at a specific ratio, degassing under vacuum and injecting the mixture into the mold set for preparing the OoC, heating the mold set for curing, then obtaining multiple layers of PDMS substrates, including a multi-channel structure after demolding, and making connection holes thereon; and
performing plasma treatment on an outer wall layer of the multiple layers of the PDMS substrates and a porous membrane, applying a PDMS adhesive around a channel, and aligning, bonding and curing the multiple layers of the PDMS substrates following a OoC structure to finally complete an assembly of the OoC that meets the design requirements.

Compared with the related art, the present invention has the following beneficial effects:

The present invention provides a method for designing OoC based on the similarity principle, a system therefor, and an OoC. All parameters of the OoC are designed from the perspectives of geometric similarity and kinetic similarity based on the similarity principle, so that the designed OoC is more similar to a real in-vivo organ in the geometric shape and fluid dynamic performance, and is therefore closer to a real state in a human body, helping improve reliability of experimental results based on the OoC. The key dimensionless number is selected as the design parameter based on actual flow characteristics of the OoC, so that the dimensionless number of the OoC is equal to a dimensionless number of the in-vivo organ, thereby achieving similar fluid dynamic performance. The key indicator parameter in the organ, for example, the shear stress on cells, is used as a design verification object, so that the OoC can more realistically and accurately simulate the in-vivo organ in terms of functions and interactions.

Some of advantages of the additional aspects of the present invention are given in the following description, and some become apparent from the following description or are learned through practice of the present invention.

### BRIEF DESCRIPTION OF THE DRAWINGS

The accompanying drawings of this specification that constitute a part of the present invention are used to provide a further understanding of the present invention. Exemplary examples of the present invention and descriptions thereof are used to explain the present invention, and do not constitute any inappropriate limitation to the present invention.

FIG. 1 is a schematic diagram of a method according to Example 1 of the present invention.

FIG. 2 is a flowchart of a method according to Example 1 of the present invention.

FIG. 3 is a schematic diagram of an entire structure of a GoC according to Example 3 of the present invention.

FIG. 4 is a schematic diagram of a channel structure of a GoC according to Example 3 of the present invention;

FIG. 5 is a cross-sectional diagram at C-C in FIG. 4.

FIG. 6 is a cross-sectional diagram at E-E in FIG. 4.

FIG. 7 is a graph of the distribution of a simulated shear stress along a D-D′ section line in FIG. 5.

FIG. 8 is a schematic diagram of simulation results of an oxygen gradient of a GoC according to Example 3 of the present invention.

FIG. 9 is a graph of an oxygen concentration gradient along a B-B′ section line in FIG. 8.

FIG. 10 is a schematic diagram of an entire structure of a LoC according to Example 4 of the present invention.

FIG. 11 is a schematic diagram of a channel structure of a LoC according to Example 4 of the present invention.

FIG. 12 is a cross-sectional diagram at F-F in FIG. 11.

FIG. 13 is a schematic diagram of simulation results of an oxygen gradient of a LoC according to Example 4 of the present invention.

FIG. 14 is a graph of the distribution of a simulated shear stress along an A-A′ section line in FIG. 13.

FIG. 15 is a graph of an oxygen concentration gradient along an A-A′ section line in FIG. 13.

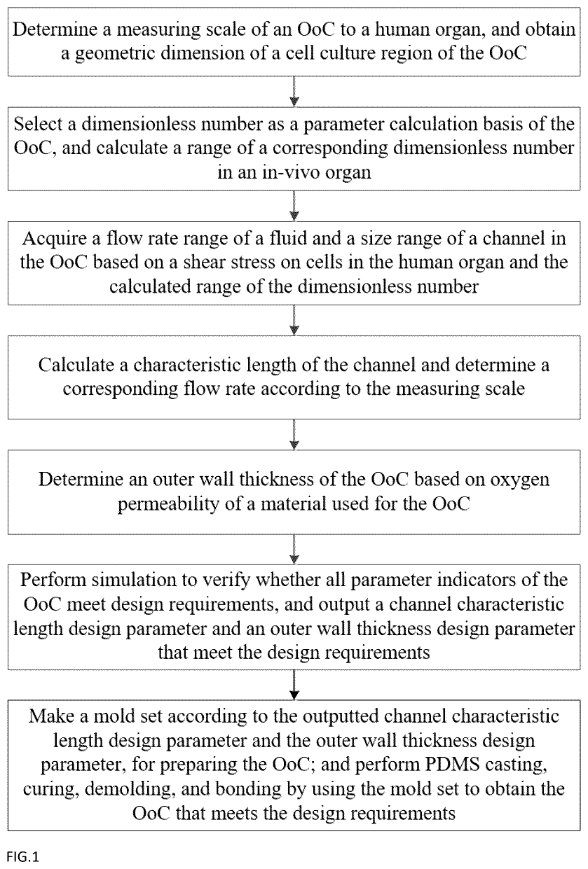

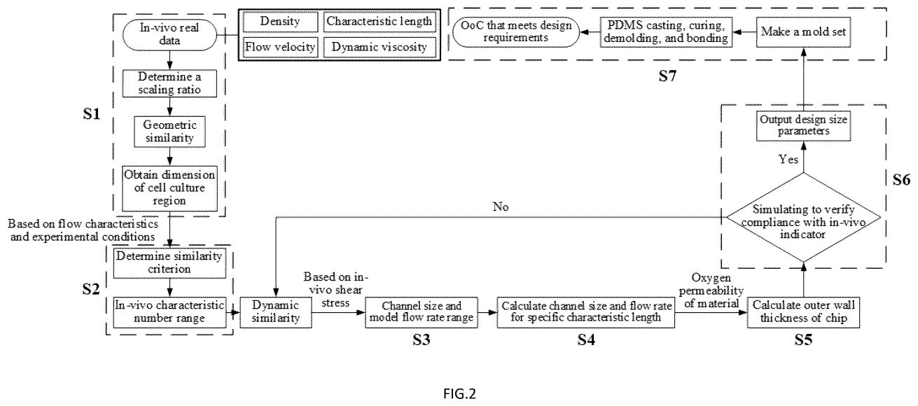

In the figures: 1-1, inlet of upper channel of GoC; 1-2, inlet of lower channel of GoC; 1-3, lower channel of GoC; 1-4, porous membrane of GoC; 1-5, outlet of upper channel of GoC; 1-6, outlet of lower channel of GoC; 1-7, dual-channel vacuum stretching cavity of GoC; 1-8, upper channel of GoC; 2-1, inlet of channel of LoC; 2-2, outlet of channel of LoC; 2-3, cell culture layer of LoC; 2-4, porous membrane of LoC; and, 2-5, channel of LoC.

### DETAILED DESCRIPTION

The following further describes the present invention with reference to the accompanying drawings and the examples.

### Example 1

The present example of the present invention provides a method for designing OoC based on the similarity principle, including the following steps:

S1: determining a scaling ratio of an OoC to a human organ, and obtaining a geometric dimension of a cell culture region of the OoC, such as the height of intestinal villi or a diameter of a hepatic lobule.

S2: selecting a dimensionless number as a parameter calculation basis of the OoC, and calculating a range of a corresponding dimensionless number in an in-vivo organ.

S3: acquiring a flow rate range of a fluid and a size range of a channel in the OoC based on a shear stress on cells in the human organ and the calculated range of the dimensionless number.

S4: calculating a characteristic length of the channel and determining a corresponding flow rate according to the scaling ratio.

S5: determining an outer wall thickness of the OoC based on the oxygen permeability of a material used for the OoC.

S6: performing a simulation to verify whether all parameter indicators of the OoC meet design requirements, and outputting a channel characteristic length design parameter and an outer wall thickness design parameter that meet the design requirements.

S7: making a mold set according to the channel characteristic length design parameter and the outer wall thickness design parameter that are outputted through calculation, for preparing the OoC; wherein, mixing PDMS and a curing agent at a specific ratio, degassing under vacuum and injecting the mixture into the mold set for preparing the OoC, heating the mold set for curing, then obtaining multiple layers of PDMS substrates including a multi-channel structure after demolding, and making connection holes thereon; and performing plasma treatment on an outer wall layer of the multiple layers of the PDMS substrates and on a porous membrane, applying a PDMS adhesive around a channel, and aligning, bonding and curing the multiple layers of the PDMS substrates following a OoC structure to finally complete an assembly of the OoC that meets the design requirements.

To make the OoC closer to a real state in the human body, the present example optimizes the OoC design steps based on the similarity principle. Based on the similarity principle, the performance or behavior of an actual object is derived from an experimental model (usually a scaled-down or scaled-up physical model). For two objects to meet the similarity principle, the two objects need to achieve geometric similarity, kinematic similarity, and dynamic similarity. Geometric similarity means that two flow fields (of a prototype and a model) have similar geometric shapes, that is, corresponding line segments are proportional in length, angles are the same, and the like. Kinematic similarity means that two flow lines are geometrically similar with the same directions and proportional magnitudes of velocity at corresponding points. Dynamic similarity means that corresponding mass points in two flows bear forces of the same type with the same directions and proportional magnitudes. Geometric similarity is the precondition of kinematic similarity and dynamic similarity. Dynamic similarity is the primary factor that determines flow similarity. Kinematic similarity is the result of geometric similarity and dynamic similarity.

To achieve similarity between the prototype and the model, geometric similarity needs to be achieved first, and then dynamic similarity needs to be achieved. To achieve dynamic similarity between two flow models, a scaling ratio of each force needs to meet a specific constraint relationship. This constraint relationship is referred to as a similarity criterion. The similarity criterion includes the Reynolds criterion (inertial force and viscous force), the Froude criterion (inertial force and gravity), the Euler criterion (inertial force and pressure), and the like.

The core to achieve dynamic similarity in the similarity principle is to keep the main dimensionless parameters equal to ensure that an experimental model and an actual object have similarity in terms of physical phenomena. The dimensionless number is a unitless number for describing a relative relationship between variables in a physical phenomenon. Common dimensionless numbers include the Strouhal number, the Froude number, the Euler number, and the Reynolds number in fluid dynamics. The Strouhal number (Sr) represents a ratio of non-motion inertial force to inertial force, defined by the equation:

$$Sr = \frac{L}{ut} ;$$

In this equation, u is a fluid velocity vector, which is, flow velocity (unit is cm/s); L is a channel characteristic length; and t is a time term.

The Froude number (Fr) is a dimensionless number for describing a ratio of gravity to inertial force in fluid dynamics, defined by the equation:

$$Fr = \sqrt{\frac{u}{gL}} ;$$

In this equation, g is the acceleration of gravity.

The Euler number (Eu) is a dimensionless number for describing a ratio of pressure to inertial force in fluid dynamics, defined by the equation:

$$Eu = \frac{p}{ρ u} ;$$

In this equation, p is fluid density (unit is g/cm³), and p is pressure (unit is Pa).

Based on the magnitudes of viscous force and inertial force on the fluid in a microchannel, the Reynolds number (Re) is defined by the equation:

$$Re = \frac{ρ uL}{μ} ;$$

In this equation, u is fluid dynamic viscosity (unit is g/cm·s).

In fluid dynamics, a fundamental equation describing fluid motion is the Navier-Stokes equation, expressed as:

$$\frac{\partial {v}_{i}}{\partial t} + {v}_{j} \frac{\partial {v}_{i}}{\partial {x}_{j}} = F - \frac{1}{ρ} \frac{\partial p}{\partial {x}_{i}} + \frac{μ}{ρ} \frac{{\partial }^{2} {v}_{i}}{\partial {x}_{j}^{2}} ;$$

In this equation, p is pressure, ρ is fluid density, μ is fluid dynamic viscosity, and F is a body force term. Herein, i is a free index that represents a physical quantity in a particular direction. For example, i=1, i=2, and i=3, respectively, correspond to components in the x, y, and z directions. In this case, vᵢ represents a component of a velocity vector in the ith direction (xi direction). Herein, j is a summation index that represents summation over all spatial directions. According to the Einstein summation convention, the index j repeated in the same expression indicates summation on j from 1 to 3 (for a three-dimensional problem). For example,

$${v}_{j} \frac{\partial {v}_{i}}{\partial {x}_{j}}$$

represents summation on the values 1, 2, and 3 of j over all directions, vⱼ is a velocity component in the j direction, and

$$\frac{\partial {v}_{i}}{\partial {x}_{j}}$$

represents a spatial change rate or a velocity component in the i direction relative to the j direction.

To transform the Navier-Stokes equation into an equation with a dimension number of 1, the reference characteristic velocity v₀, the reference characteristic length L₀, the reference characteristic time t₀ and the reference characteristic pressure p₀ are introduced. Quantities with a dimension number of 1 are defined as follows:

$${v}_{i}^{0} = {v}_{i} / {v}_{0} ;$$

$${x}_{i}^{0} = {x}_{i} / {L}_{0} ;$$

$${t}^{0} = t / {t}_{0} ;$$

$${p}^{0} = p / {p}_{0} ;$$

wherein, vᵢ⁰ represents dimensionless velocity,

$${x}_{i}^{0}$$

represents dimensionless time, t⁰ represents dimensionless time, and p⁰ represents dimensionless pressure.

The characteristic quantities (the dimensionless velocity

$${v}_{i}^{0} ,$$

the dimensionless position

$${x}_{i}^{0} ,$$

the dimensionless time t⁰, and the dimensionless pressure p⁰) are put into the Navier-Stokes equation to be transformed into the equation with the dimension number of 1:

$$\frac{{L}_{0}}{{v}_{0} {t}_{0}} \frac{\partial {v}_{i}^{0}}{\partial {t}^{0}} + {v}_{j}^{0} \frac{\partial {v}_{i}^{0}}{\partial {x}_{j}^{0}} = \frac{1}{\frac{{v}_{0}^{2}}{{gL}_{0}}} - \frac{{p}_{0}}{ρ {v}_{0}^{2}} \frac{\partial {p}^{0}}{\partial {x}_{i}^{0}} + \frac{1}{\frac{{v}_{0} ρ {L}_{0}}{μ}} \frac{{\partial }^{2} {v}_{i}^{0}}{\partial {x}_{j}^{02}} ;$$

wherein,

$$\frac{{L}_{0}}{{v}_{0} {t}_{0}}$$

is the Strouhal number,

$$\sqrt{\frac{{v}_{0}^{2}}{{gL}_{0}}}$$

is the Froude number,

$$\frac{{p}_{0}}{ρ {v}_{0}^{2}}$$

is the Euler number, and

$$\frac{{v}_{0} ρ {L}_{0}}{μ}$$

is the Reynolds number. The four dimensionless numbers are put into the equation to obtain a relation equation between the fluid kinematic parameter and the dimensionless number:

$$Sr \frac{\partial {v}_{i}^{0}}{\partial {t}^{0}} + {v}_{j}^{0} \frac{\partial {v}_{i}^{0}}{\partial {x}_{j}^{0}} = \frac{1}{{Fr}^{2}} - Eu \frac{\partial {p}^{0}}{\partial {x}_{i}^{0}} + \frac{1}{Re} \frac{{\partial }^{2} {v}_{i}^{0}}{\partial {x}_{j}^{02}} .$$

However, it is very difficult to ensure that all the dimensionless numbers are the same as those of the human organism. Therefore, when an actual flow field is solved, the four dimensionless numbers in the equation are compared to determine that the key dimensionless numbers are the same. In the case of steady flow, the Sr number may differ. If gravity in the flow field has a small effect compared with other forces, the Fr number may be ignored. If pressure in the flow field is constant, the Eu number may be ignored. Therefore, in the design of the OoC, the Reynolds criterion is the primary focus, because the magnitude of the Reynolds number has a direct effect on the calculation of the Navier-Stokes equation. When the Reynolds number is less than 1, viscous force dominates, and the effect on the flow field may be ignored.

As microchannels of the OoC are mostly of rectangular structures, the characteristic length L may be calculated using the following equation:

$$L = \frac{2 WH}{W + H} ;$$

wherein, W is a channel width, and H is a channel height.

In addition, the shear stress generated by the fluid in the channel significantly affects cell growth, differentiation, and functions. To ensure that the shear stress in the chip is comparable to that in the human organ and more accurately simulate the physiological state of cells in vivo, to improve the reliability of experimental data, according to the law of internal friction of Newtonian fluid dynamics, the shear stress on the cells is solved as:

$$τ = \frac{6 μ Q}{{wH}^{2}} ;$$

wherein, τ is fluid shear stress (unit is dyne/cm²), and Q is a volumetric flow rate (unit is cm³/s).

Finally, when an outer wall thickness of the OoC is determined, the oxygen flux N_{O}_{₂} of the material used for the OoC is calculated to represent the oxygen permeability of the material used:

$${N}_{{O}_{2}} = {K}_{0} ( {p}_{{O}_{2}} - {k}_{{O}_{2}} {c}_{{O}_{2}} ) ;$$

wherein, K₀ is an overall mass transfer coefficient (K₀=Pₘ/σ, where Pₘ is the oxygen permeability of the material, and σ is the thickness of the material), p_{O}_{₂} is the oxygen partial pressure in an incubator, k_{O}_{₂} is the Henry's law constant of oxygen 9.901×10⁻⁶ mol/(m³·Pa), and c_{O}_{₂} is the oxygen concentration. In the present example, the material used for the OoC is polydimethylsiloxane (PDMS), and corresponding oxygen permeability is Pₘ=5.1×10⁻¹⁸ (mol·m)/m²·s·Pa.

In step S1, to facilitate the manufacturing of the OoC and cell culture, the scaling ratio of the OoC to the human organ is determined. A scaled-down GoC is designed for an organ with a geometric dimension larger than that of the OoC, for example, the intestine. A scaled-up LoC is designed for an organ with a microstructure, for example, a hepatic lobule. The scaling ratio may be determined based on a proportional relationship between an OoC designed in the related art and an organ structure, and the organ structure may be scaled down according to the scaling ratio to obtain the geometric dimension of the cell culture region of the OoC.

In step S2, the dimensionless number includes a Strouhal number, a Froude number, an Euler number, and a Reynolds number. The dimensionless number that has the greatest influence on the fluid flow in the OoC is selected as the calculation basis. The Reynolds criterion is usually selected as the calculation basis in consideration of the actual state of the fluid flow field in the organ. To be specific, calculation is performed based on consistency of the Reynolds number of the designed OoC and the Reynolds number of the corresponding organ, to ensure similarity between the OoC and the in-vivo organ in terms of fluid dynamic performance. Therefore, it is necessary to calculate a range of a corresponding in-vivo Reynolds number of the organ based on in-vivo data of the organ.

In step S3, considering that the shear stress significantly affects cell growth, differentiation, and functions, to realistically reproduce flow characteristics of the fluid in the organ and accurately simulate the physiological state of cells in vivo, the magnitude of the shear stress on the cells in the organ and the calculated range of the dimensionless number are put into the relation equation between the fluid kinematic parameter and the dimensionless number, the relation equation and the calculation equation of the characteristic length of the channel of the OoC are solved simultaneously to obtain the size range of the channel, and the corresponding flow velocity and flow rate of the fluid in the channel are also calculated.

In step S4, the characteristic length of the channel of the OoC is obtained based on the characteristic length of the organ under study with the scaling ratio determined in step S1, and then the design parameters of the channel, such as the height, the width, the flow velocity, the volumetric flow rate, and the dimensionless number, are calculated.

In step S5, a weak oxygen gradient is formed in the OoC due to oxygen consumption by cell respiration when the cell culture region in the OoC is in an initial state. However, due to a relatively high oxygen permeability of the PDMS material, the oxygen flux outside the OoC is greater than the oxygen consumption rate of the cells, making it difficult to reproduce the oxygen gradient in the chip. To establish an oxygen gradient in the OoC similar to that in vivo, first, based on the oxygen permeability of the PDMS material, the oxygen concentration range in the OoC is simulated, and the thickness of the wall of the OoC is controlled; then, the surface of the chip is coated with a membrane of a highly airtight material (for example, polyvinylidene chloride (PVDC)) to control permeation of oxygen from the external environment, so that the oxygen concentration or oxygen gradient in the OoC is close to that in the in-vivo environment.

In step S6, the designed OoC is verified by simulation for the shear stress and the oxygen concentration. The magnitude of the shear stress, the range of the oxygen concentration, and the oxygen gradient in the channel of the OoC are obtained through the simulation to determine whether the OoC meets the design requirements, and the design size parameter that is of the OoC and that meets the design requirements is outputted, such as the channel characteristic length design parameter and the outer wall thickness design parameter.

In step S7, the mold set for preparing the OoC is made according to the channel characteristic length design parameter and the outer wall thickness design parameter that are outputted through calculation; PDMS and the curing agent are mixed at a specific ratio, the mixture is degassed under vacuum and injected into the mold set for preparing the OoC, the mold set is heated for curing, and then demolding is performed to obtain the multiple layers of PDMS substrates including the channel structure, and the connection holes are made; and plasma treatment is performed on the outer wall layer of the multiple layers of PDMS substrates and the porous membrane, the PDMS adhesive is applied around the channel, and the multiple layers of the PDMS substrates are aligned, bonded, and cured following a OoC structure, to finally complete an assembly of the OoC that meets the design requirements.

### Example 2

This example provides a system for designing OoC based on the similarity principle, including:
a geometric similarity module, configured to determine a scaling ratio of an OoC to a human organ, and obtain a geometric dimension of a cell culture region of the OoC;
a dimensionless number selection module, configured to select a dimensionless number as a parameter calculation basis of the OoC, and calculate a range of a corresponding dimensionless number in an in-vivo organ;
a flow rate and channel size calculation module, configured to acquire a flow rate range of a fluid and a size range of a channel in the OoC based on a shear stress on cells in the human organ and the calculated range of the dimensionless number;
a characteristic length calculation module, configured to calculate a characteristic length of the channel and determine a corresponding flow rate according to the scaling ratio;
an outer wall thickness calculation module, configured to determine an outer wall thickness of the OoC based on oxygen permeability of a material used for the OoC; and a verification module, configured to perform simulation to verify whether all parameter indicators of the OoC meet design requirements, and output a channel characteristic length design parameter and an outer wall thickness design parameter that meet the design requirements;
wherein, a mold set for preparing the OoC is made according to the channel characteristic length design parameter and the outer wall thickness design parameter that are outputted through calculation; and

PDMS casting, curing, demolding, and bonding are performed by using the mold set to obtain the OoC that meets the design requirements.

Specifically, mixing PDMS and a curing agent at a specific ratio, degassing under vacuum and injecting the mixture into the mold set for preparing the OoC, heating the mold set for curing, then obtaining multiple layers of PDMS substrates including a multi-channel structure after demolding, and making connection holes thereon; and performing plasma treatment on an outer wall layer of the multiple layers of the PDMS substrates and a porous membrane, applying a PDMS adhesive around a channel, and aligning, bonding and curing the multiple layers of the PDMS substrates following a OoC structure to finally complete an assembly of the OoC that meets the design requirements.

It should be noted herein that the modules in this example are in a one-to-one correspondence with the steps of the method in Example 1, with the same specific implementation processes. Details are not described herein again.

### Example 3

In the present example, a GoC is designed and prepared with specific parameters based on the method for designing the OoC based on the similarity principle provided in Example 1.

A mold set for preparing the GoC is made according to specific design parameters outputted through calculation, such as a channel characteristic length and an outer wall thickness of the GoC. PDMS and a curing agent are mixed at a specific ratio, the mixture is degassed under vacuum and injected into the mold set for preparing the GoC, the mold set is heated for curing, and then demolding is performed to obtain multiple layers of PDMS substrates having channel structures, and then connection holes are made. Plasma treatment is performed on an outer wall layer of the multiple layers of PDMS substrates and a porous membrane, a PDMS adhesive is applied around a microchannel, and the multiple layers of PDMS substrates are aligned, bonded, and cured following a GoC structure, to finally complete assembly of the GoC that meets the design requirements.

An optimization design of the GoC is primarily based on the structure and function of the intestine, with the microfluidic technology, aiming to simulate a complex physiological environment in the intestine. First, the geometric design should mimic the three-dimensional morphology of the intestine as much as possible, especially the intestinal villus structure, to maximize the surface area for absorption of nutrients and drugs. In addition, precise control of the flow velocity and shear stress is critical to reproduce the flow characteristics in different regions of the intestine to promote growth, differentiation, and functional maintenance of epithelial cells.

An optimization target of the design of the present example is to subject the cells to the shear stress of 0.0002 Pa to 0.008 Pa and to maintain the cells under the oxygen partial pressure of 10 mmHg to 59 mmHg.

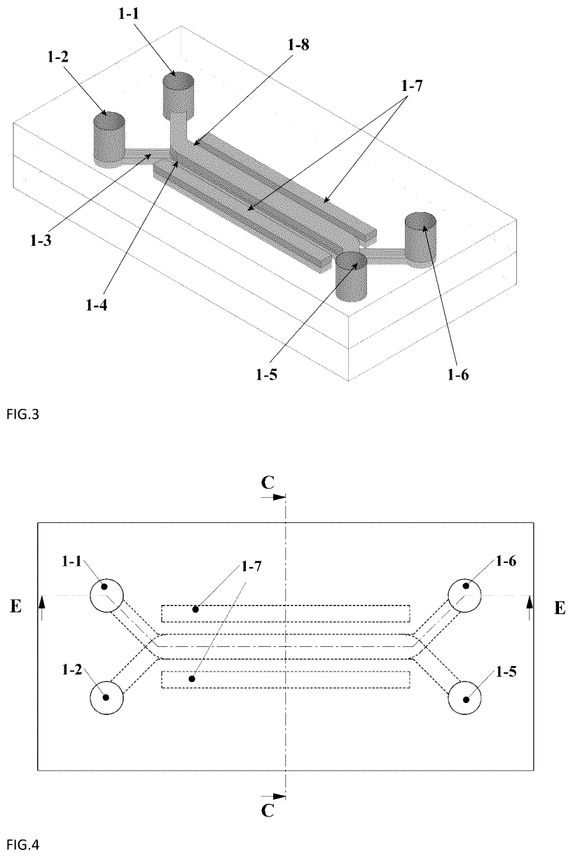

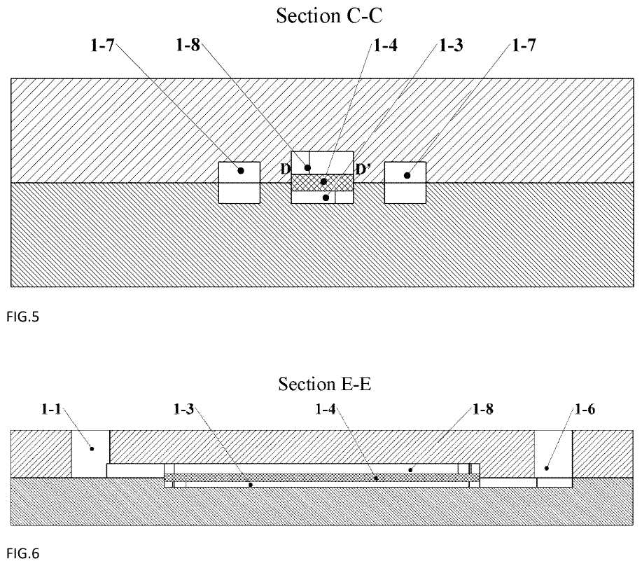

As shown in FIG. 3 to FIG. 6, the structure of the designed GoC includes an inlet of an upper channel of the GoC 1-1, an inlet of a lower channel of the GoC 1-2, the lower channel of GoC 1-3, a porous membrane of GoC 1-4, an outlet of the upper channel of the GoC 1-5, an outlet of the lower channel of the GoC 1-6, a dual-channel vacuum stretching cavity of the GoC 1-7, and the upper channel of GoC 1-8. Wherein, the upper channel of the GoC 1-8 simulates an intestinal lumen environment, where intestinal epithelial cells are seeded, and a culture medium is poured to provide nutrients for cell growth and the shear stress. The lower channel of the GoC 1-3 simulates an intestinal vascular environment, where a culture medium is poured to remove metabolites of the intestinal epithelial cells. The upper channel of the GoC 1-8 and the lower channel of the GoC 1-3 are separated by the porous membrane of the GoC 1-4 covered with an extracellular matrix (ECM), allowing substance exchange between cells, and maintaining the microenvironments similar to the inner walls and external blood vessels of the intestine respectively above and below the porous membrane of the GoC 1-4. The GoC dual-channel vacuum stretching cavities 1-7 are provided on two sides of the GoC channels. The air pressure in the stretching cavities on the two sides is changed to generate cyclic stretching on the porous membrane of the cultured cells, to simulate intestinal peristalsis.

Viscosity of chyme in the intestine is a complex parameter affected by various factors, including food composition, digestion stages, individual differences, and the like. Through measurement and analysis of the viscosity of chyme under different conditions, the viscosity of chyme is 0.01 Pa·s to 1 Pa·s, the density is 1,000 kg/m³ to 1,400 kg/m³, and the flow velocity is 0.005 m/s to 0.02 m/s. In addition, the average diameter of the small intestine is 0.025 m.

The villus sizes in experiments of GoCs in the related art are investigated. In some experiments, it is found that the villus-like height is 40 μm to 50 μm on day 3 of the experiment. In some other experiments, the measured villus-like height is 37.04±2.38 μm. The villus height in the human intestine is about 500 μm. Based on this, the scale determined in this example is 12.5. According to the actual size of the intestinal villi with the determined scale, the villus height, diameter, and spacing are scaled down, the villus density is kept unchanged, and the scaled-down geometric dimension is determined, as shown in Table 1.

TABLE 1
Geometric dimension of the villus structure in the GoC

| | | |
|---|---|---|
| Villus size | Actual size | Scaled-down size |

| | | | | |
|---|---|---|---|---|
| Length | 500 | μm | 40 | μm |
| Diameter | 160 | μm | 12.8 | μm |
| Spacing | 40 | μm | 3.2 | μm |

| | | |
|---|---|---|
| Density | 15.8 ± 0.5/mm² | 15.8 ± 0.5/mm² |

Considering that the fluid flow in the GoC is primarily affected by viscous force and inertial force, the Reynolds criterion is selected as the calculation basis for flow similarity, that is, the Reynolds number is selected as the dimensionless number for the parameter calculation basis.

The range of the Reynolds number of the small intestine in vivo is calculated based on in-vivo data. A lower limit value Reₘᵢₙ of the Reynolds number is obtained under the conditions of minimum flow velocity, minimum density, and maximum viscosity:

$${Re}_{min} = \frac{{ρ}_{min} {u}_{min} L}{{μ}_{max}} = \frac{1000 \times 0.005 \times 0.025}{1} = 0.1 \; 25 ;$$

wherein, ρₘᵢₙ represents minimum chyme density, uₘᵢₙ represents minimum chyme flow velocity, L represents a characteristic length, which is the average diameter 0.025 m of the small intestine, and μₘₐₓ represents maximum chyme viscosity.

An upper limit value Reₘₐₓ of the Reynolds number is obtained under the conditions of maximum flow velocity, maximum density, and minimum viscosity:

$${Re}_{max} = \frac{{ρ}_{max} {u}_{max} L}{{μ}_{min}} = \frac{1400 \times 0.02 \times 0.025}{0.01} = 70 ;$$

wherein, ρₘₐₓ represents maximum chyme density, uₘₐₓ represents maximum chyme flow velocity, and μₘᵢₙ represents the minimum chyme viscosity.

Studies have shown that, when intestinal epithelial cells are subjected to a shear stress of 0.02 dyne/cm²=0.002 Pa=0.02 g/(cm·s²), polarization can be accelerated, and spontaneous formation of a folded cell monolayer can be promoted, forming a villus-like structure. Assuming that the density of the culture medium is 1,000 kg/m³=1 g/cm³, the viscosity of the culture medium is 0.001 Pa·s=0.01 g/(cm·s), and the channel width is twice the channel height, the foregoing parameters are put into the Reynolds number calculation equation, the characteristic length calculation equation, and the shear stress calculation equation, and then the equations are solved simultaneously to obtain:

$$Re = \frac{400 uH}{3} ;$$

$$\frac{u}{H} = \frac{1}{3} ;$$

The range of the Reynolds number is put into the foregoing equations to obtain the size range of the channel. When the Reynolds number is the lower limit value 0.125, through calculation, the channel height is 0.053 cm, the channel width is 0.106 cm, the flow velocity is 0.0177 cm/s, and the volumetric flow rate is 0.053 cm×0.106 cm×0.0177 cm/s=0.0000994386 cm³/s (0.3579 L/h).

When the Reynolds number is the upper limit value of 70, through corresponding calculation, the channel height is 1.255 cm, the channel width is 2.51 cm, the flow velocity is 0.41833 cm/s, and the volumetric flow rate is 1.31776 cm³/s (4,743,937.649 μL/h).

In conclusion, the size range of the channel includes the channel height 0.053 cm to 1.255 cm and the channel width 0.106 cm to 2.51 cm; and the range of the volumetric flow rate is 0.0000994386 cm³/s to 1.31776 cm³/s (0.3579 μL/h to 4,743,937.649 μL/h).

From the in-vivo data, the average diameter of the human small intestine is 25 mm. Since only one side of the villi to the intestinal center is studied, the characteristic length of the small intestine is 12.5 mm in this example, and the characteristic length of the GoC is scaled down to 1 mm according to the scale 12.5. Assuming that the channel width is twice the channel height, through calculation, the corresponding channel height is 0.75 mm, and the corresponding channel width is 1.5 mm. In this case, the flow velocity is 0.25 mm/s, the volumetric flow rate is 0.28125 mm³/s=1012.5 μL/h, and the Reynolds number is 0.25.

The material used for the GoC is PDMS. A permeability coefficient of oxygen in PDMS is 5.8×10⁻¹⁰ cm³ (STP) cm/cm²·s·Pa. A permeability coefficient of oxygen in a PVDC membrane on the surface of the chip is 1×10⁻¹⁶ cm³ (STP) cm/cm²·s·Pa. The oxygen concentration range is simulated to make the oxygen concentration in the GoC close to that in the in-vivo environment, to determine the outer wall thickness of the GoC. According to the simulation results, a distance from the upper channel to the top of the chip is 3.5 mm, and a distance from the lower channel to the bottom of the chip is 1 mm.

The obtained parameter indicators of the GoC are shown in Table 2. The oxygen partial pressure range is obtained according to the simulation results.

TABLE 2
Design parameters of the GoC

| | In-vivo | GoC designed in |
|---|---|---|
| | intestine data | the example |
| Channel characteristic | Diameter - 25 mm | Channel width is |
| size | | 1.06 to 25.1 (mm) |
| | | Channel height is |
| | | 0.53 to 12.55 (mm) |
| Reynolds number | 0.125-70 | 0.25 |
| Shear stress | 0.0002 to 0.008 (Pa) | 0.002 Pa |
| Oxygen partial | 10 to 59 (mmHg) | 22 to 35 (mmHg) |
| pressure range | | |

According to the determined characteristic length 1 mm of the GoC, the corresponding channel height is 0.75 mm, the corresponding channel width is 1.5 mm, the flow velocity is 0.25 mm/s, the volumetric flow rate is 0.28125 mm³/s=1012.5 μL/h, and the Reynolds number is 0.25. Simulation is performed on the GoC designed using the foregoing parameters to verify whether the shear stress and oxygen concentration range meet the design requirements.

The results of the shear stress distribution in the channel obtained by computational fluid dynamics simulation are shown in FIG. 7. In this case, the shear stress on cells is 0.002 Pa to 0.003 Pa, which matches the in-vivo data.

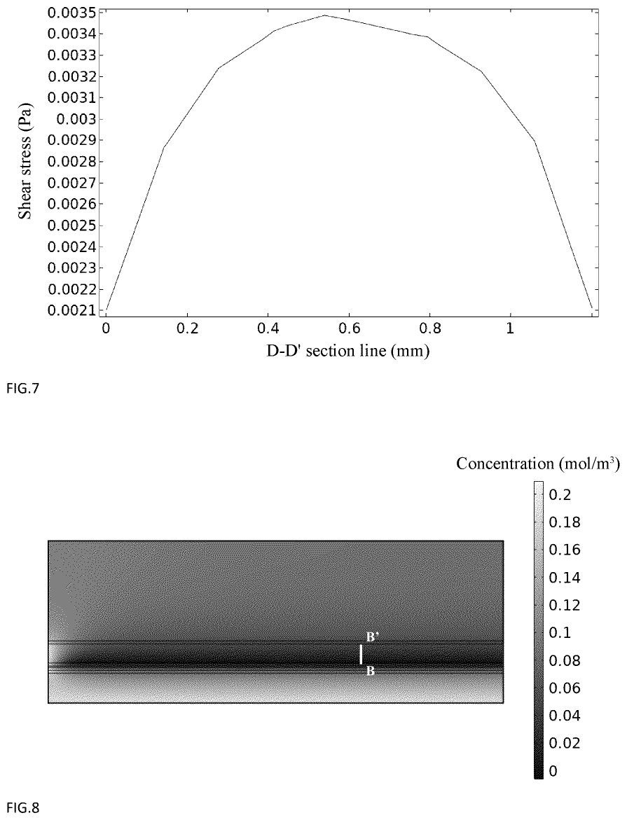

The oxygen concentration range in the GoC is shown in FIG. 8 and FIG. 9. An oxygen gradient of 22 mmHg to 35 mmHg is formed in the upper channel.

The molds for preparing the GoC are designed and made according to the channel characteristic length and the outer wall thickness of the GoC, and PDMS casting, curing, demolding, and bonding are performed to obtain the GoC that meets the design requirements.

### Example 4

In the present example, a LoC is designed and prepared with specific parameters based on the method for designing the OoC based on the similarity principle provided in Example 1, to simulate the structure and function of the liver to reproduce in vitro physiological processes of the liver, such as metabolism, detoxification, and secretion.

A mold set for preparing the LoC is made according to specific design parameters outputted through calculation, such as a channel characteristic length and an outer wall thickness of the LoC. PDMS and a curing agent are mixed at a specific ratio, the mixture is degassed under vacuum and injected into the mold set for preparing the LoC, the mold set is heated for curing, and then demolding is performed to obtain multiple layers of PDMS substrates including a channel structure, and then connection holes are made. Plasma treatment is performed on an outer wall layer of the multiple layers of the PDMS substrates and a porous membrane, a PDMS adhesive is applied around a channel, and the multiple layers of the PDMS substrates are aligned, bonded, and cured following a LoC structure, to finally complete assembly of the LoC that meets the design requirements.

The geometric design of the LoC should mimic a microscopic hexagonal structure of a hepatic lobule. Then, a microchannel is constructed in the chip by the microfluidic technology to ensure that fluid dynamics conditions in the chip are similar to characteristics of blood flow in the liver. The control of the fluid flow velocity, shear stress, and flow state is crucial. The flow in the chip should be kept in a laminar state to maintain the normal function and metabolic activity of hepatocytes.

An optimization target of this example is to ensure that an oxygen concentration gradient similar to that in an in-vivo hepatic sinusoid structure is formed in the LoC, that is, from 65 mmHg at the periphery of the chip to 30 mmHg. Studies have shown that a shear stress of 4.7×10⁻⁴ Pa can reduce CYP1A2 expression of hepatocytes to 1/20. Therefore, the shear stress on cells in the designed LoC should be as small as possible (it is better to be close to 0 Pa).

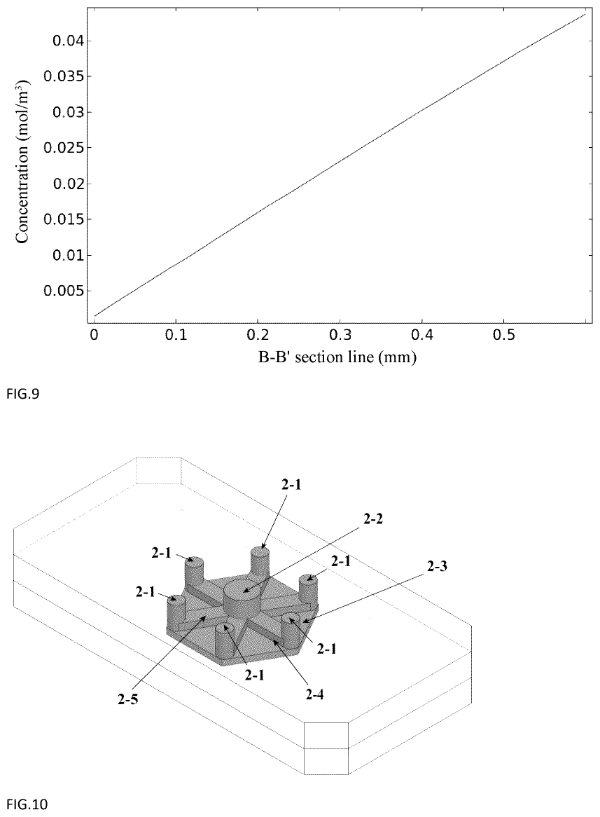

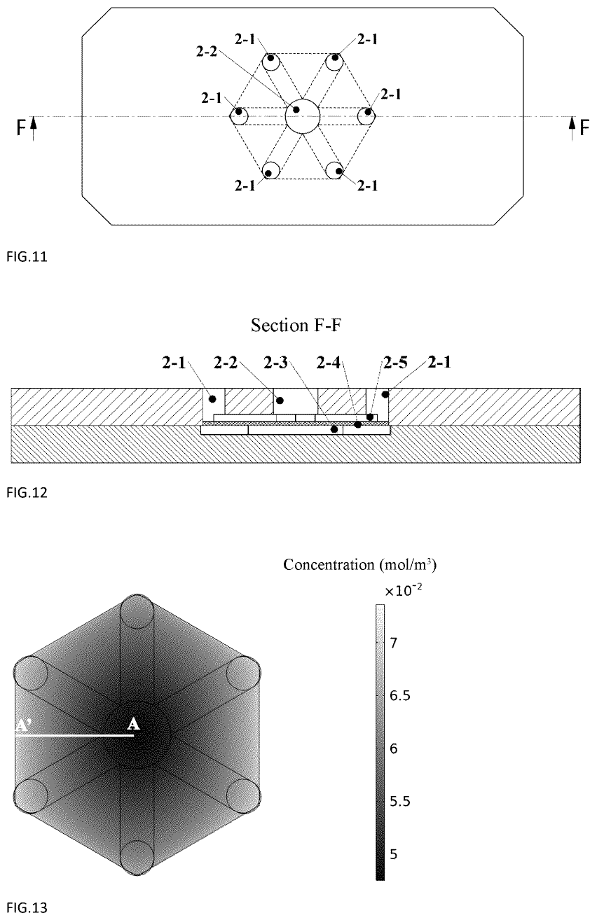

The structure of the designed LoC is shown in FIG. 10 to FIG. 12. Six evenly distributed channels of the LoC 2-5 are designed on the upper layer of the chip. Inlets of the six channels of the LoCs are separated, and outlets of the six channels of the LoCs converge. Each of the six channels has an inlet of the channel of the LoC 2-1. The six channels share one outlet of the channel of the LoC 2-2 provided in the center. The lower portion of the six channels of the LoCs is provided with a cell culture layer of the LoC 2-3. The structure of the cell culture layer of the LoC is close to the in-vivo structure. The cell culture layer of the LoC and the channels of the LoCs are separated by a porous membrane of the LoC 2-4. A culture medium is poured into the six channels of the LoCs on the upper layer to simulate a flow path from the portal vein to the central vein. The geometric structure of the cell culture layer of the LoC on the lower layer is close to the hexagonal in-vivo hepatic lobule, and the culture medium flows from the channels on the upper layer through the porous membrane of the LoC. The structure reproduces blood flow characteristics resulting from the window permeation effect of hepatic sinusoids.

An adult liver contains about 0.5 to 1 million hepatic lobules and 1 billion hepatic sinusoids. A hepatic lobule is a polygonal prism with a diameter of about 1 mm. One hepatic lobule contains 1,000 to 2,000 hepatic sinusoids. In addition, the diameter of a hepatic sinusoid vessel is about 0.01 mm. Some studies have found that red blood cells in hepatic sinusoids move by 107.43 μm within 302 ms, based on which a calculated velocity of blood flow in the hepatic sinusoids is 354.4 μm/s. Some other studies provide that a blood density range is 1,043 kg/m³ to 1,060 kg/m³, and a blood viscosity range is 3×10⁻³ Pa·s to 5×10⁻³ Pa·s.

A hepatic lobule is a polygonal prism with a diameter of about 1 mm. This size is too small to make a LoC at 1:1. To facilitate manufacturing, in this example, the scale is determined as 5 for scaling up, so that the cell culture region in the OoC is a hexagonal structure with a diameter of 5 mm.

Considering that the fluid flow in the LoC is primarily affected by viscous force and inertial force, the Reynolds criterion is selected as the calculation basis for flow similarity, that is, the Reynolds number is selected as the dimensionless number for the parameter calculation basis.

Based on in-vivo parameters, the cross-sectional area A of a single hepatic sinusoid is first calculated:

$$A = \frac{π {D}^{2}}{4} = \frac{π \times 0. 01 \times 0.01}{4} = 7.85 \times 1 {0}^{- 5} {mm}^{2} ;$$

wherein, D is the diameter of a hepatic sinusoid vessel.

The hepatic sinusoid vessels in the liver lobule are interconnected, and it is estimated that there are about 1,500 hepatic sinusoid vessels in one hepatic lobule. To facilitate manufacturing of the OoC, in this example, 1500 channels are equated to six OoC microchannels. In other words, the flow area of each of the OoC channels is equal to the total flow area of 250 hepatic sinusoid vessels, and the cross-sectional area of each of OoC microchannels is 250×7.85×10⁻⁵ mm²=0.019625 mm².

Then, the characteristic length of the in-vivo channel is calculated according to the formula for the area of a circle:

$$0.019625 = \frac{π {L}^{2}}{4} ;$$

Through calculation, the characteristic length of the in-vivo channel is L=0.158 mm=1.58×10⁻⁴ m.

The Reynolds number range in the six simplified channels is calculated according to the Reynolds number equation. A lower limit value of the Reynolds number is obtained through calculation under the conditions of minimum blood density and maximum blood viscosity, which is 0.0116. An upper limit value of the Reynolds number is obtained through calculation under the conditions of maximum blood density and minimum blood viscosity, which is 0.0197.

Studies have shown that a shear stress of 4.7×10−4 Pa can reduce CYP1A2 expression of cells to 1/20, and the survival rate of hepatocytes decreases to 25% when the shear stress reaches 0.1 Pa. To avoid this problem, the hepatocytes are cultured on the lower layer of the LoC and are protected from the negative effects of the shear stress through the porous membrane. Therefore, it is only necessary to solve a proportional relationship between the flow rate and the channel size of the model according to the relation equation between the fluid kinematic parameter and the dimensionless number. It is assumed that the density of the culture medium is 1,000 kg/m³=1 g/cm³, the viscosity of the culture medium is 0.001 Pa·s=0.01 g/(cm·s), and the channel width is twice the channel height. When the Reynolds number is the lower limit value 0.0166, through calculation, a numerical relationship between the flow velocity u and the channel height H is u·H=0.000087 (cm). When the Reynolds number is the upper limit value 0.0197, a numerical relationship between the flow velocity and the channel height is u·H=0.00014775 (cm).

According to a scale of the characteristic length to the length of the in-vivo channel, the characteristic length of the LoC is 1.58×10⁻⁴ m×5=7.9×10⁻⁴ m=0.79 mm, and then through calculation, the channel height is 0.5925 mm, and the channel width is 1.185 mm.

Finally, through calculation, when the Reynolds number is the lower limit value 0.0166, the flow velocity is 0.01468 mm/s, the channel cross-sectional area is 0.5925 mm×1.185 mm=0.7021125 mm², and the volumetric flow rate is 0.014683 mm/s×0.702112 mm²=0.010309 mm³/s=0.010309 μL/s=37.1128 μL/h.

When the Reynolds number is the upper limit value 0.0197, the flow velocity is 0.0249 mm/s, and the volumetric flow rate is 0.0249 mm/s×0.7021125 mm²=0.01748 mm³/s=0.0175 μL/s=63.03 μL/h.

In conclusion, the channel height is 0.5925 mm, and the channel width is 1.185 mm. In this case, the flow velocity range is 0.001468 mm/s to 0.0249 mm/s, and the volumetric flow rate range is 37.1128 μL/h to 63.03 μL/h.

The oxygen concentration range is simulated to make the oxygen concentration in the LoC close to that in vivo, to determine the outer wall thickness of the LoC. According to the simulation results, a distance from the upper channel to the top of the chip is 1.4 mm, and a distance from the lower channel to the bottom of the chip is 1.5 mm.

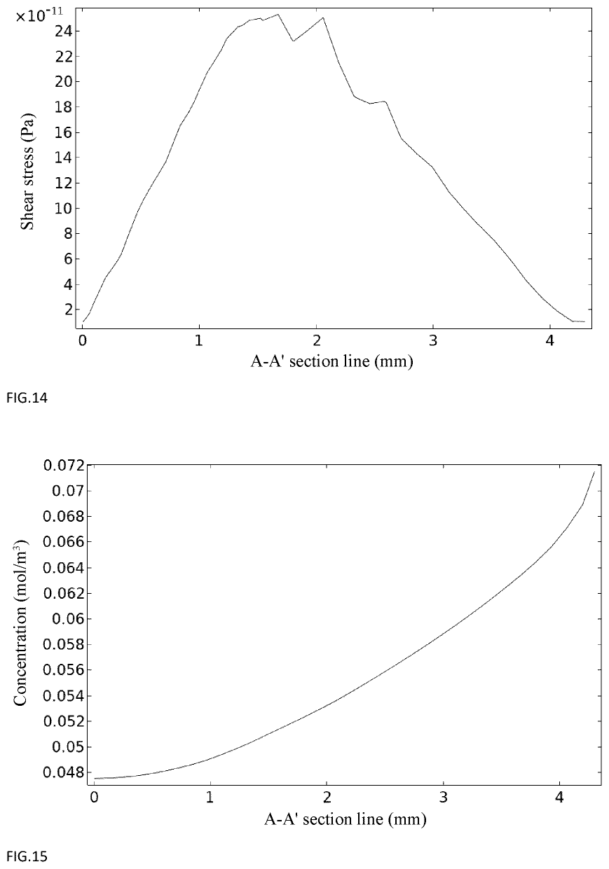

Simulation is performed on the designed LoC, where the channel height of the LoC is 0.5925 mm, the channel width is 1.185 mm, the flow velocity is 0.0249 mm/s, the volumetric flow rate is 63.03 μL/h, and the Reynolds number is 0.0197. The simulation results of the shear stress are shown in FIG. 13. In this case, the shear stress on cells is 2.5×10⁻¹⁰ Pa, which is close to 0 and achieves the expected effect. The simulation results of the oxygen concentration gradient are shown in FIG. 14 and FIG. 15. The oxygen concentration gradient is 35.6 mmHg to 55 mmHg, which matches the in-vivo data.

The foregoing descriptions are merely preferred embodiments of the present invention, but are not intended to limit the present invention. A person skilled in art may make various alterations and variations to the present invention. Any modification, equivalent replacement, or improvement made within the spirit and principle of the present invention shall fall within the protection scope of the present invention.

1. A method for manufacturing an organ-on-a-chip (OoC) based on a similarity principle, the method comprising:
determining a scaling ratio of an OoC to be designed to a human organ, and obtaining a geometric dimension of a cell culture region of the OoC to be designed;
selecting a Reynolds number (dimensionless number) as a calculation basis of design parameters of the OoC to be designed, then calculating a range of the Reynolds number in an in-vivo organ;
acquiring a flow rate range of a fluid and a size range of the channels in the OoC to be designed based on a shear stress on cells in the in-vivo organ and the calculated range of the Reynolds number, comprises:
acquiring a relation equation between a fluid kinematic parameter and the Reynolds number based on a Navier-Stokes equation;
calculating the flow rate range of the fluid and the size range of the channels in the OoC to be designed by using the relation equation, a shear stress on cells in the OoC to be designed, and the range of the Reynolds number in the in-vivo organ, comprises:
setting a ratio of a channel width to a channel height of the channels to be 2:1, then putting the shear stress on the cells in the OoC to be designed, density and viscosity of a culture medium in the cell culture region, and the ratio of the channel width to the channel height into a Reynolds number calculation equation, a characteristic length calculation equation, and a shear stress calculation equation, then solving the three equations simultaneously, and putting the range of the Reynolds number in the in-vivo organ into the three simultaneous equations to obtain the size range of the channels based on the ratio of the channel width to the channel height and the flow rate range of the fluid in the OoC to be designed;
calculating a characteristic length of the channels and determining a corresponding flow rate according to the scaling ratio;
determining an outer wall thickness of the OoC to be designed based on an oxygen permeability of polydimethylsiloxane (PDMS) material;
verifying whether all parameter indicators of the OoC to be designed meet design requirements by using a simulation; and outputting a verified characteristic length of the channels as the design parameter of the channel characteristic length and a verified design parameter of the outer wall thickness of the OoC to be designed;
making a mold set for manufacturing the OoC according to the verified characteristic length of the channels and the verified design parameter of the outer wall thickness;
mixing the PDMS material and a curing agent at a specific ratio to obtain a mixture, degassing the mixture under vacuum and injecting the mixture into the mold set;
after the injection, heating the mold set for curing the mixture;
after the mixture is cured, demolding the mold set to obtain multiple layers of PDMS substrates containing a multi-channel structure, and making connection holes on the PDMS substrates;
performing a plasma treatment on an outer wall layer and a porous membrane of the multiple layers of the PDMS substrates, applying a PDMS adhesive around channels; and
aligning and bonding the multiple layers of the PDMS substrates, and curing the bonded PDMS substrates to obtain the OoC.

2. The method according to claim 1, wherein obtaining a geometric dimension of a cell culture region of the OoC comprises: scaling down an organ structure according to the scaling ratio to obtain the geometric dimension of the cell culture region of the OoC.

3. (canceled)

4. The method according to claim 1, wherein the Reynolds number is selected as the calculation basis of the design parameters of the OoC to be designed based on an actual state of a flow field of the fluid in the in-vivo organ.

5. The method according to claim 1, the method further comprising: coating a surface of the OoC with a polyvinylidene chloride (PVDC) membrane to control a permeation of oxygen from an external environment.

6. (canceled)

7. An organ-on-a-chip (OoC), being manufactured by using the method according to claim 1.

8. The OoC according to claim 7, being a gut-on-a-chip (GoC) and comprising an upper channel of the GoC for simulating an intestinal lumen environment and a lower channel of the GoC for simulating an intestinal vascular environment, wherein:
the upper channel of the GoC and the lower channel of the GoC are separated by a porous membrane of the GoC covered with an extracellular matrix; a width of the upper channel is twice a height of the upper channel, and a width of the lower channel is twice a height of the lower channel; and
the GoC further comprises GoC dual-channel vacuum stretching cavities on two sides of the upper channel of the GoC and the lower channel of the GoC, for simulating intestinal peristalsis by changing air pressure.

9. The OoC according to claim 7, being a liver-on-a-chip (LoC) and comprising a plurality of channels of the LoC that are evenly distributed, each of the plurality of the channels of the LoC comprises an inlet of channel of the LoC, the plurality of the channels of the LoCs share one outlet of channel of the LoC, a lower portion of the plurality of the channels of the LoC is provided with a cell culture layer of the LoC, and the cell culture layer of the LoC and the plurality of the channels of the LoC are separated by a porous membrane of the LoC; wherein, a width of the plurality of the channels of the LoC is twice a height of the plurality of the channels of the LoC.
